(19) United States
(12) Patent Application Publication
(10) Pub. No.: US 2002/0111596 A1
(43) Pub. Date: Aug. 15, 2002

(54) Garment having removable side panels

(76) Inventors: Amy L. Fletcher, Appleton, WI (US); David Arthur Kuen, Neenah, WI (US); Robert Lee Popp, Hortonville, WI (US); Jack L. Couillard, Menasha, WI (US); Joseph D. Coenen, Neenah, WI (US); Christopher Peter Olson, Neenah, WI (US); Shawn A. Quereshi, Neenah, WI (US)

Correspondence Address: Melanie I. Rauch, Pauley Petersen Kinne & Fejer, Suite 365, 2800 West Higgins Road, Hoffman Estates, IL, 60195, US

(21) Appl. No.: 09/784,292

(22) Filed: Feb. 15, 2001

(51) Int. Cl. A61F 13/15

(52) U.S. Cl. 604/385.03; 604/385.22; 604/387; 604/391

(57) ABSTRACT

A pant-like absorbent garment having removable side panels that are releasable and refastenable, such that the resulting garment can be opened and closed on either a front side of the garment or a back side of the garment on either a left side or a right side of the garment. Furthermore, the removable side panels can be reused with a disposable chassis portion of the garment.

### FIELD OF THE INVENTION

This invention is directed to pant-like, personal care absorbent products having removable side panels that are releasable and refastenable.

### BACKGROUND OF THE INVENTION

Pant-like absorbent garments, such as adult incontinence wear as well as infant and children's diapers, swim wear and training pants, typically have adhesive or mechanical fasteners on the sides for donning and removal, or else rely on a stretchable waist opening and leg openings to slide on and off the wearer. Adhesive fasteners wear out in as little as one use. Therefore, if a care giver checks the status of the wearer's absorbent garment contents by unfastening an adhesive fastener, the garment often must be replaced due to a worn out adhesive fastener even if the absorbent garment itself is not in need of changing.

Absorbent garments that slide on and off a wearer are often messy after use. Furthermore, in order to remove such absorbent garments, the wearer's clothing covering the absorbent garments, such as pants, must generally be completely removed. Checking the status of the wearer's absorbent garment contents is often just as cumbersome as changing the absorbent garment.

Another drawback to the current design of absorbent garments is the amount of waste produced by each garment. Many components of absorbent garments can be made of materials that can be flushed in toilets or are otherwise biodegradable, but certain components, particularly elastomeric components, should be durable and thus may not be flushable in toilets. Without elastomeric components, pant-like absorbent garments typically cannot conform to a wearer's body to provide comfort and leakage protection.

There is a need or desire for pant-like, personal care absorbent garments that have refastenable side seams for ease of removal and donning without complete removal of a wearer's clothing.

### SUMMARY OF THE INVENTION

The present invention is directed to pant-like absorbent garments having side panels that are releasably attached to a chassis portion of the garment such that the side panels can be unfastened from the garment, refastened to the garment, and removed from the garment altogether. The resulting absorbent garments have refastenable side seams and can be easily fitted to and removed from a wearer without complete removal of a wearer's clothing.

The refastenable side seams extend from a waist opening to each of two leg openings, attaching a front panel to the side panels, and attaching a back panel to the side panels. Each of the refastenable side seams includes a fastening component and a mating fastening component, each of which suitably includes either a hook material or a loop material. More particularly, a first side panel includes first and second fastening components, a second side panel includes third and fourth fastening components, the front panel includes first and third mating fastening components engageable with the first and third fastening components, and the back panel includes second and fourth mating fastening components engageable with the second and fourth fastening components.

Furthermore, the chassis portion of the garment can be flushable, or otherwise biodegradable, while the removable side panels can be reusable, thereby reducing the amount of waste materials per garment. More particularly, the elastomeric components of the garment can be included in the side panels rather than on the chassis, such that the chassis can be made of all flushable materials while the elastomeric components of the side panels provide conformity of the garment to the wearer's body. In addition, since the side panels are reusable, the amount of waste, both flushable and non-flushable, is reduced in general.

Alternative embodiments of the invention include an elastomeric chassis with removable non-elastomeric side panels; an elastomeric chassis with removable elastomeric side panels; and either an elastomeric or a non-elastomeric chassis with removable side panels that are partially elastomeric.

With the foregoing in mind, it is a feature and advantage of the invention to provide a pant-like absorbent garment that can be easily applied to and easily removed from a wearer without the need to entirely remove the wearer's clothing. It is another feature and advantage of the invention to provide a pant-like absorbent garment including a flushable chassis portion and reusable side panels.

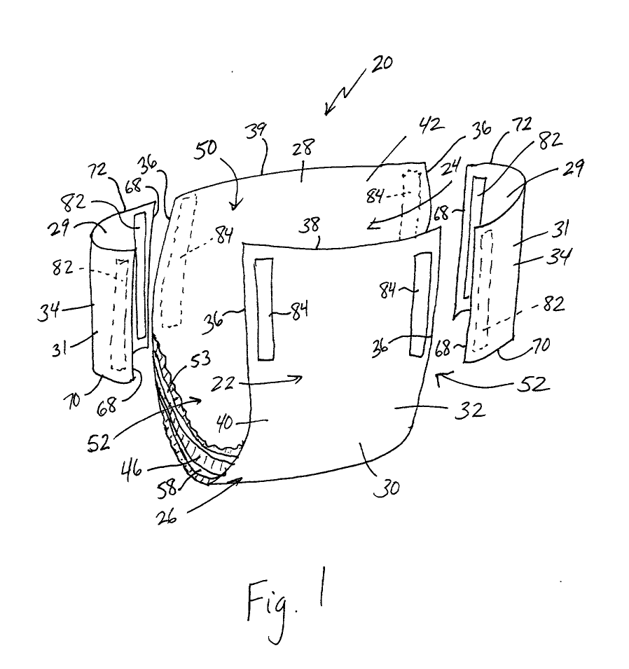

FIG. 1 is a perspective view of an absorbent garment having removable side panels;

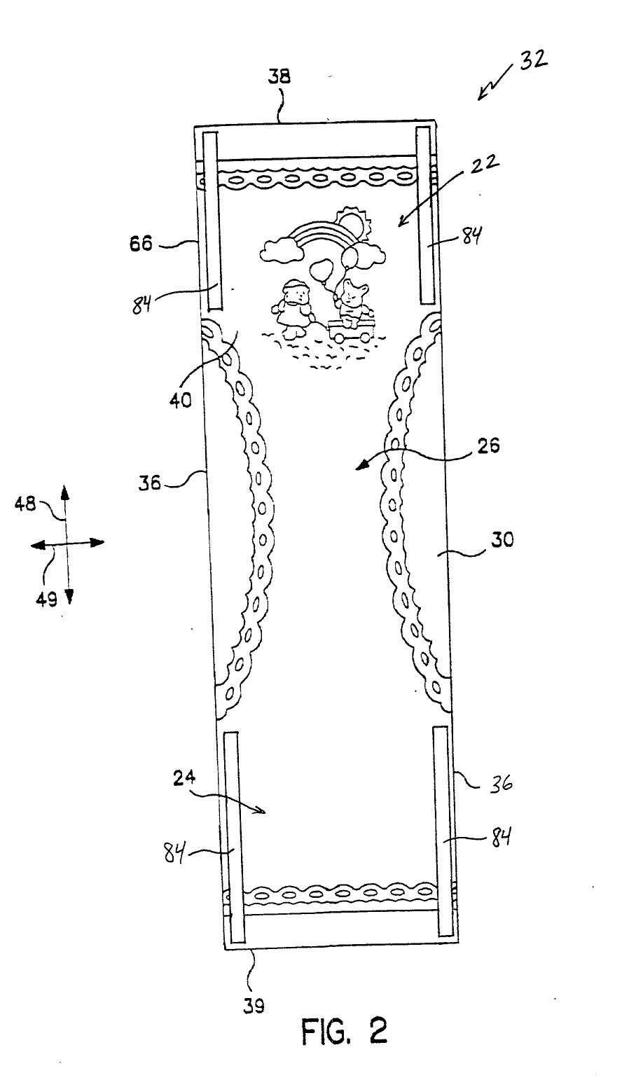

FIG. 2 is a plan view of a chassis of an absorbent garment in a stretched flat state showing the surface of the garment that faces away from the wearer when the garment is worn;

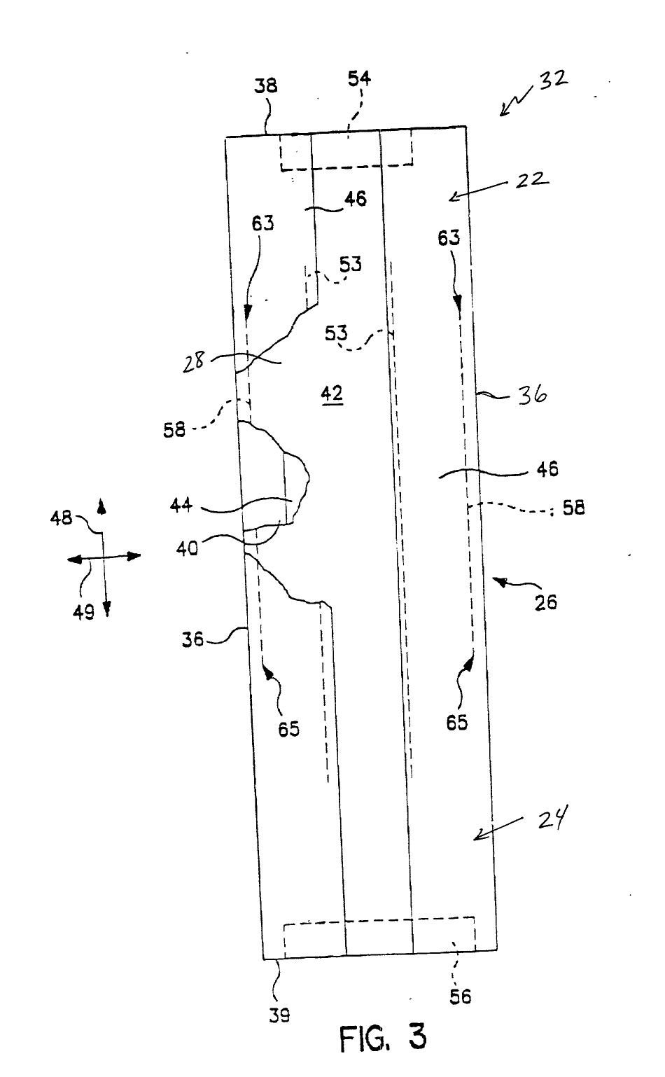

FIG. 3 is a plan view of a chassis of an absorbent garment in a stretched flat state showing the surface of the garment that faces the wearer when the garment is worn, and with portions cut away to show the underlying features;

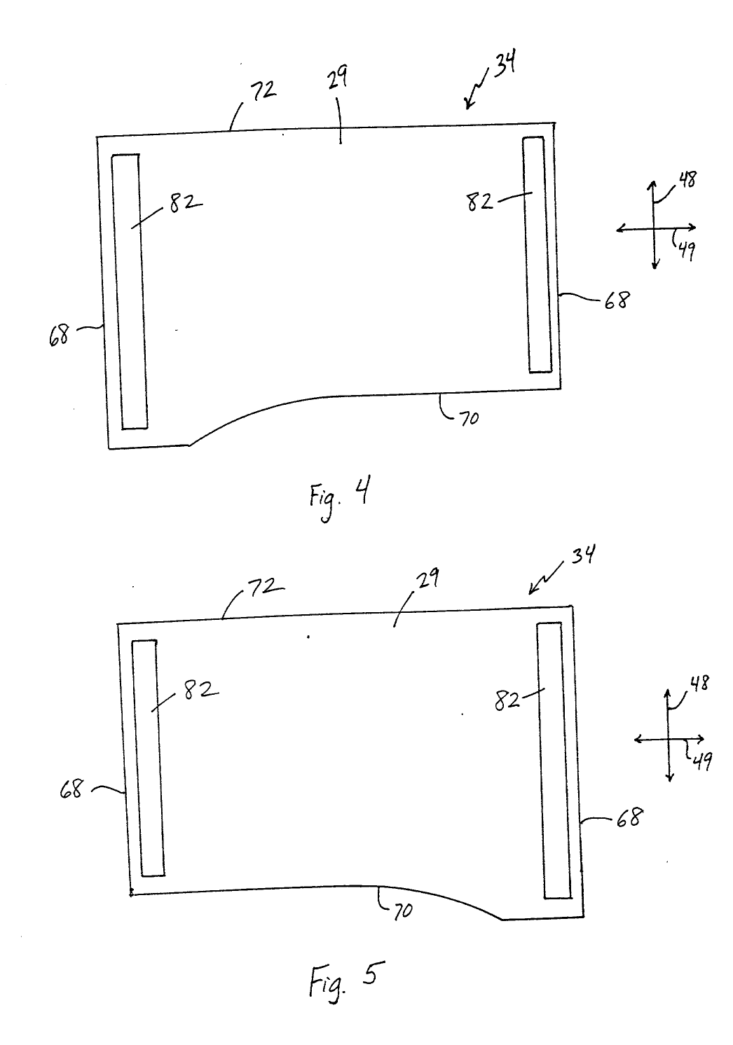

FIG. 4 is a plan view of a left side panel of an absorbent garment in a stretched flat state, showing the surface of the panel that faces the wearer when the garment is worn;

FIG. 5 is a plan view of a right side panel of an absorbent garment in a stretched flat state, showing the surface of the panel that faces the wearer when the garment is worn;

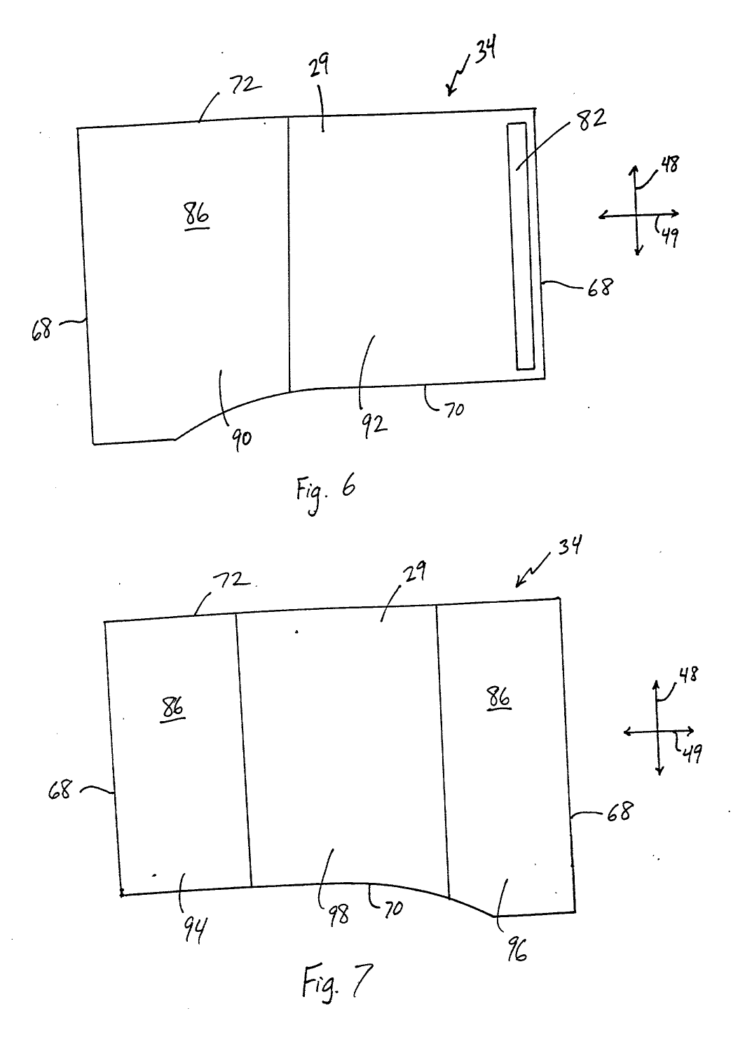

FIG. 6 is a plan view of another embodiment of a left side panel of an absorbent garment in a stretched flat state, showing the surface of the panel that faces the wearer when the garment is worn;

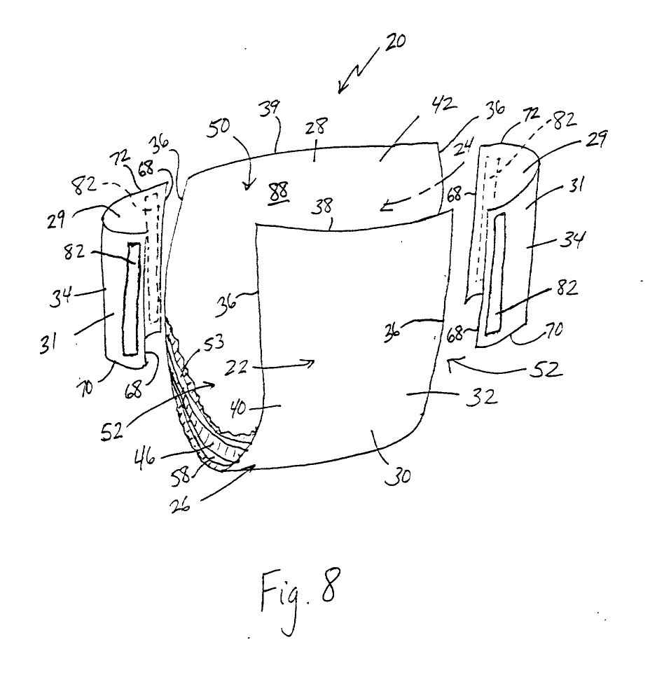

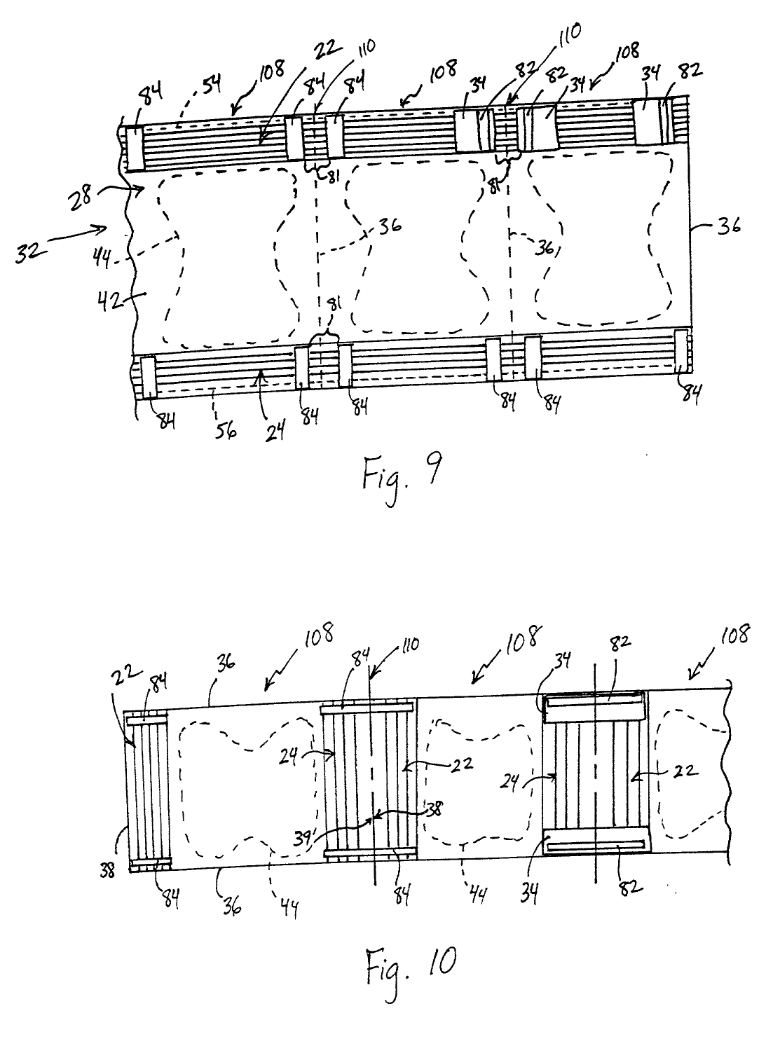

FIG. 7 is a plan view of another embodiment of a right side panel of an absorbent garment in a stretched flat state, showing the surface of the panel that faces the wearer when the garment is worn;

FIG. 8 is a perspective view of another embodiment of an absorbent garment having removable side panels;

FIG. 9 is a plan view of a cross direction assembly for producing an absorbent garment having removable side panels;

FIG. 10 is a plan view of a machine direction assembly for producing an absorbent garment having removable side panels;

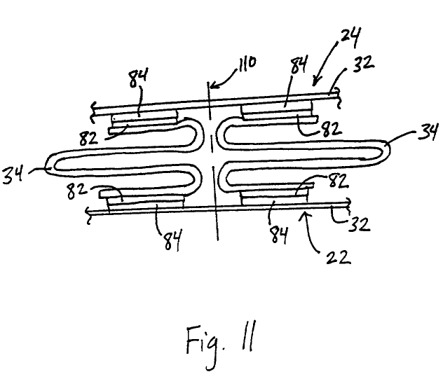

FIG. 11 is a top view of fastening systems of two adjacent garments having removable side panels during assembly of the garments;

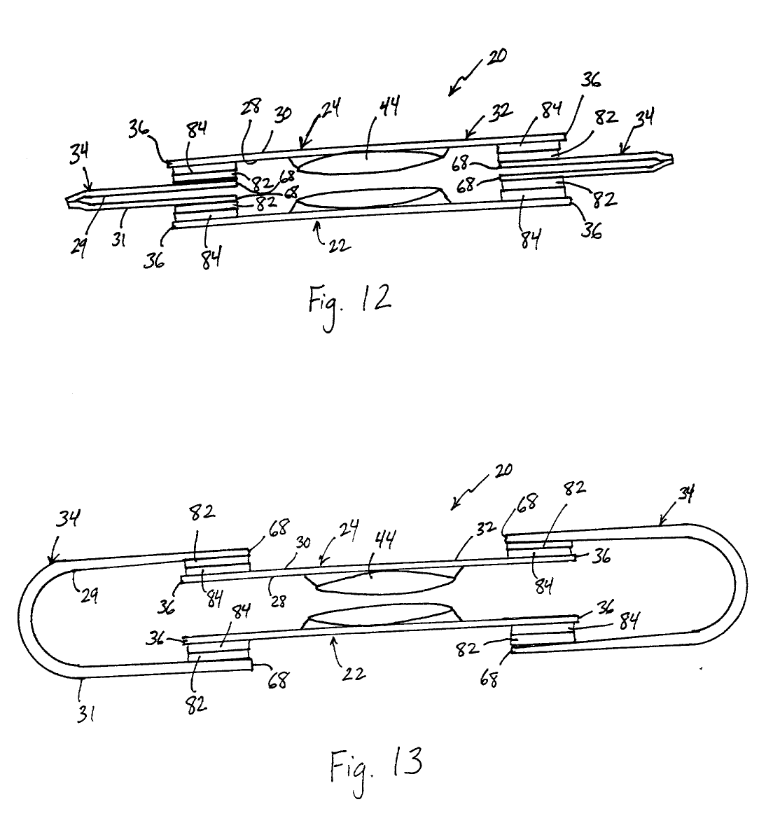

FIG. 12 is a top view of one embodiment of an absorbent garment having removable side panels;

FIG. 13 is a top view of another embodiment of an absorbent garment having removable side panels;

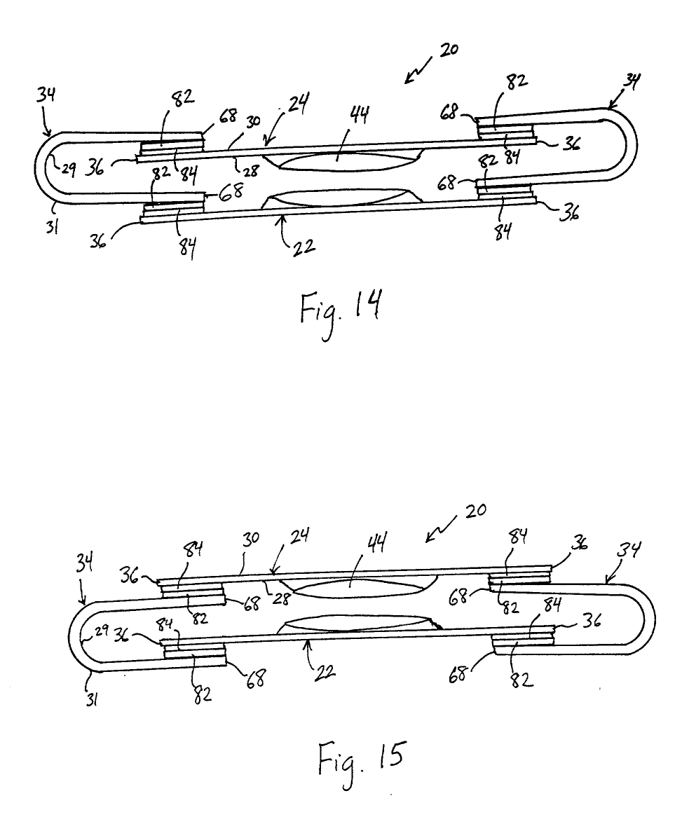

FIG. 14 is a top view of yet another embodiment of an absorbent garment having removable side panels; and

FIG. 15 is a top view of still another embodiment of an absorbent garment having removable side panels.

### DEFINITIONS

Within the context of this specification, each term or phrase below will include the following meaning or meanings.

“Bonded” refers to the joining, adhering, connecting, attaching, or the like, of at least two elements. Two elements will be considered to be bonded together when they are bonded directly to one another or indirectly to one another, such as when each is directly bonded to intermediate elements.

“Connected” refers to the joining, adhering, bonding, attaching, or the like, of at least two elements. Two elements will be considered to be connected together when they are connected directly to one another or indirectly to one another, such as when each is directly connected to intermediate elements.

“Cross direction” refers to the width of a fabric in a direction generally perpendicular to the direction in which it is produced, as opposed to “machine direction” which refers to the length of a fabric in the direction in which it is produced.

“Disposable” refers to articles which are designed to be discarded after a limited use rather than being laundered or otherwise restored for reuse. As used herein, the term “disposable” can include flushable, biodegradable and compostable articles.

“Durable” refers to articles which are designed to be reused an unlimited number of times for the same purpose.

“Elastomeric” and “elastic” refer to that property of a material or composite by virtue of which it tends to recover its original size and shape after removal of a force causing a deformation. It is generally preferred that the elastomeric material or composite be capable of being elongated by at least 50 percent, more preferably by at least 300 percent, of its relaxed length and recover, upon release of an applied force, at least 50 percent of its elongation.

“Film” refers to a thermoplastic film made using a film extrusion process, such as a cast film or blown film extrusion process. The term includes apertured films, slit films, and other porous films which constitute liquid transfer films, as well as films which do not transfer liquid.

“Flushable” refers to materials that can be flushed down a toilet by virtue of the material's tendency to dissolve, disintegrate, biodegrade, rupture and/or otherwise disperse in water.

“Hydrophilic” describes fibers or the surfaces of fibers which are wetted by the aqueous liquids in contact with the fibers. The degree of wetting of the materials can, in turn, be described in terms of the contact angles and the surface tensions of the liquids and materials involved. Equipment and techniques suitable for measuring the wettability of particular fiber materials or blends of fiber materials can be provided by a Cahn SFA-222 Surface Force Analyzer System, or a substantially equivalent system. When measured with this system, fibers having contact angles less than 90 are designated “wettable” or hydrophilic, while fibers having contact angles greater than 90 are designated “nonwettable” or hydrophobic.

“Layer” when used in the singular can have the dual meaning of a single element or a plurality of elements.

“Liquid impermeable,” when used in describing a layer or multi-layer laminate, means that a liquid, such as urine, will not pass through the layer or laminate, under ordinary use conditions, in a direction generally perpendicular to the plane of the layer or laminate at the point of liquid contact.

“Liquid permeable material” or “liquid water-permeable material” refers to a material present in one or more layers, such as a film, nonwoven fabric, or open-celled foam, which is porous, and which is water permeable due to the flow of water and other aqueous liquids through the pores. The pores in the film or foam, or spaces between fibers or filaments in a nonwoven web, are large enough and frequent enough to permit leakage and flow of liquid water through the material.

“Longitudinal” and “transverse” have their customary meaning, as indicated by the longitudinal and transverse axes depicted in FIGS. 2-7. The longitudinal axis lies in the plane of the article and is generally parallel to a vertical plane that bisects a standing wearer into left and right body halves when the article is worn. The transverse axis lies in the plane of the article generally perpendicular to the longitudinal axis. The article as illustrated is longer in the longitudinal direction than in the transverse direction.

“Machine direction” refers to the lengthwise direction of a fabric in the direction in which it is produced, as opposed to “cross direction” which refers to the widthwise direction of a fabric, generally perpendicular to the machine direction.

“Meltblown fiber” means fibers formed by extruding a molten thermoplastic material through a plurality of fine, usually circular, die capillaries as molten threads or filaments into converging high velocity heated gas (e.g., air) streams which attenuate the filaments of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed for example, in U.S. Pat. No. 3,849,241 to Butin et al. Meltblown fibers are microfibers which may be continuous or discontinuous, are generally smaller than about 0.6 denier, and are generally self bonding when deposited onto a collecting surface. Meltblown fibers used in the present invention are preferably substantially continuous in length.

“Member” when used in the singular can have the dual meaning of a single element or a plurality of elements.

“Nonwoven” and “nonwoven web” refer to materials and webs of material which are formed without the aid of a textile weaving or knitting process.

“Operatively joined,” in reference to the attachment of an elastic member to another element, means that the elastic member when attached to or connected to the element, or treated with heat or chemicals, by stretching, or the like, gives the element elastic properties; and with reference to the attachment of a non-elastic member to another element, means that the member and element can be attached in any suitable manner that permits or allows them to perform the intended or described function of the joinder. The joining, attaching, connecting or the like can be either directly, such as joining either member directly to an element, or can be indirectly by means of another member disposed between the first member and the first element.

“Permanently bonded” refers to the joining, adhering, connecting, attaching, or the like, of two elements of an absorbent garment such that the elements tend to be and remain bonded during normal use conditions of the absorbent garment.

“Polymers” include, but are not limited to, homopolymers, copolymers, such as for example, block, graft, random and alternating copolymers, terpolymers, etc. and blends and modifications thereof Furthermore, unless otherwise specifically limited, the term “polymer” shall include all possible geometrical configurations of the material. These configurations include, but are not limited to isotactic, syndiotactic and atactic symmetries.

“Refastenable” refers to the property of two elements being capable of releasable attachment, separation, and subsequent releasable reattachment without substantial permanent deformation or rupture.

“Releasably attached,” “releasably engaged” and variations thereofrefer to two elements being connected or connectable such that the elements tend to remain connected absent a separation force applied to one or both of the elements, and the elements being capable of separation without substantial permanent deformation or rupture. The required separation force is typically beyond that encountered while wearing the absorbent garment.

“Spunbonded fiber” refers to small diameter fibers which are formed by extruding molten thermoplastic material as filaments from a plurality of fine capillaries of a spinnerette having a circular or other configuration, with the diameter of the extruded filaments then being rapidly reduced as by, for example, in U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, U.S. Pat. No. 3,502,538 to Petersen, and U.S. Pat. No. 3,542,615 to Dobo et al., each of which is incorporated herein in its entirety by reference. Spunbond fibers are quenched and generally not tacky when they are deposited onto a collecting surface. Spunbond fibers are generally continuous and often have average deniers larger than about 0.3, more particularly, between about 0.6 and 10.

“Stretchable” means that a material can be stretched, without breaking, to at least 150% of its initial (unstretched) length in at least one direction, suitably to at least 200% of its initial length, desirably to at least 250% of its initial length.

“Superabsorbent” or “superabsorbent material” refers to a water-swellable, water-insoluble organic or inorganic material capable, under the most favorable conditions, of absorbing at least about 15 times its weight and, more desirably, at least about 30 times its weight in an aqueous solution containing 0.9 weight percent sodium chloride. The superabsorbent materials can be natural, synthetic and modified natural polymers and materials. In addition, the superabsorbent materials can be inorganic materials, such as silica gels, or organic compounds such as cross-linked polymers.

“Surface” includes any layer, film, woven, nonwoven, laminate, composite, or the like, whether pervious or impervious to air, gas, and/or liquids.

“Thermoplastic” describes a material that softens when exposed to heat and which substantially returns to a nonsoftened condition when cooled to room temperature.

These terms may be defined with additional language in the remaining portions of the specification.

### DETAILED DESCRIPTION OF THE PRESENTLY PREFERRED EMBODIMENTS

The present invention is directed to a pant-like absorbent garment having removable side panels that are releasable and refastenable. The removable side panels can be reused with a flushable chassis portion of the garment. Furthermore, the removable side panels provide ease of removal and donning of the absorbent garment without complete removal of a wearer's clothing.

The principles of the present invention can be incorporated into any suitable pant-like disposable absorbent article. Examples of such suitable articles include diapers, training pants, incontinence products, other personal care or health care garments, or the like. As used herein, the term “incontinence products” includes absorbent underwear for children, absorbent garments for children or young adults with special needs such as autistic children or others with bladder/bowel control problems as a result of physical disabilities, as well as absorbent garments for incontinent older adults. For ease of explanation, the description hereafter will be in terms of a child's training pant.

Referring to FIG. 1, a disposable absorbent article, such as a training pant 20, is illustrated in an unfastened condition. The training pant 20 includes an absorbent chassis 32 and a pair of removable side panels 34. The absorbent chassis 32 defines a front region 22, a back region 24, a crotch region 26 interconnecting the front and back regions, an inner surface 28 which is configured to contact the wearer, and an outer surface 30 opposite the inner surface which is configured to contact the wearer's clothing. With additional reference to FIGS. 2 and 3, the absorbent chassis 32 also defines a pair of transversely opposed distal edges 36 and a pair of longitudinally opposed waist edges, which are designated front waist edge 38 and back waist edge 39. The front region 22 is contiguous with the front waist edge 38, and the back region 24 is contiguous with the back waist edge 39. For reference, arrows 48 and 49 depicting the orientation of the longitudinal axis and the transverse axis, respectively, of the training pant 20 are illustrated in FIGS. 2-7.

The illustrated absorbent chassis 32 includes an outer cover 40, a bodyside liner 42 which is connected to the outer cover in a superposed relation, an absorbent assembly 44 which is located between the outer cover 40 and the bodyside liner 42, and a pair of containment flaps 46, as shown in FIG. 3. The removable side panels 34, shown in FIGS. 4-7, each include fastening components incorporated therein, either in the form of separate fastening components 82, as shown in FIGS. 4 and 5, or in the form of fastening material 86 forming at least a portion of the side panels, as shown in FIGS. 6 and 7, such that one fastening component on each side panel 34 can be releasably engaged with a mating fastening component incorporated into the front region 22 of the chassis 32 and one fastening component on each side panel 34 can be releasably engaged with a mating fastening component 84 incorporated into the back region 24 of the chassis 32. Similarly, the mating fastening components can be in the form of either separate mating fastening components 84, as shown in FIGS. 1 and 2, or in the form of mating fastening material 88 forming at least a portion of the chassis, as shown in FIG. 8. In various embodiments, either the entire outer cover 40 or the entire body side liner 42 can be made of a mating fastening material.

With the training pant 20 in the fastened position, as can be derived from FIG. 1, the front and back regions 22 and 24 are indirectly connected to one another by the two side panels 34 to define a three-dimensional pant configuration having a waist opening 50 and a pair of leg openings 52. The front region 22 includes the portion of the training pant 20 which, when worn, is positioned on the front of the wearer while the back region 24 includes the portion of the training pant which, when worn, is positioned on the back of the wearer. The crotch region 26 of the training pant 20 includes the portion of the training pant which, when worn, is positioned between the legs of the wearer and covers the lower torso of the wearer. The side panels 34 include the portions of the training pant 20 which, when worn, are positioned on the hips of the wearer.

As shown in FIG. 3, the front region 22 of the absorbent chassis 32 can include a front waist elastic member 54 and any other connected components. The back region 24 of the absorbent chassis 32 can include a rear waist elastic member 56 and any other connected components. The waist edges 38 and 39 of the absorbent chassis 32, indirectly connected to one another by the side panels 34, are configured to encircle the waist of the wearer when worn and provide the waist opening 50 which defines a waist perimeter dimension. Portions of the transversely opposed distal edges 36 of the chassis 32 in the crotch region 26, along with leg end edges 70 of the side panels 34, generally define the leg openings 52.

The absorbent chassis 32 is configured to contain and/or absorb any body exudates discharged from the wearer. For example, the absorbent chassis 32 desirably, although not necessarily, includes the pair of containment flaps 46 which are configured to provide a barrier to the transverse flow of body exudates. A flap elastic member 53 (FIG. 3) can be operatively joined with each containment flap 46 in any suitable manner as is well known in the art. The elasticized containment flaps 46 define an unattached edge which assumes an upright, generally perpendicular configuration in at least the crotch region 26 of the training pant 20 to form a seal against the wearer's body. The containment flaps 46 can be located along the transversely opposed distal edges 36 of the absorbent chassis 32, and can extend longitudinally along the entire length of the absorbent chassis or may only extend partially along the length of the absorbent chassis. Suitable constructions and arrangements for the containment flaps 46 are generally well known to those skilled in the art and are described in U.S. Pat. No. 4,704,116 issued Nov. 3, 1987 to Enloe, which is incorporated herein by reference.

To further enhance containment and/or absorption of body exudates, the training pant 20 can include the front waist elastic member 54, the rear waist elastic member 56, and leg elastic members 58, as are known to those skilled in the art (FIG. 3). The waist elastic members 54 and 56 can be operatively joined to the outer cover 40 and/or bodyside liner 42 along the opposite waist edges 38 and 39, and can extend over part or all of the waist edges. The leg elastic members 58 can be operatively joined to the outer cover 40 and/or bodyside liner 42 while longitudinally aligned along the distal edges 36 and positioned in the crotch region 26 of the chassis 32. Each leg elastic member 58 has a front terminal point 63 and a back terminal point 65, which points represent the longitudinal ends of the elastic gathering caused by the leg elastic members. The front terminal points 63 are desirably located adjacent the longitudinally innermost parts of the mating fastening components 84 in the front region 22 of the chassis 32, and the back terminal points 65 are desirably located adjacent the longitudinally innermost parts of the mating fastening components 84 in the back region 24 of the chassis 32.

The flap elastic members 53, the waist elastic members 54 and 56, and the leg elastic members 58 can be formed of any suitable elastic material. As is well known to those skilled in the art, suitable elastic materials include sheets, strands or ribbons of natural rubber, synthetic rubber, or thermoplastic elastomeric polymers. The elastic materials can be stretched and adhered to a substrate, adhered to a gathered substrate, or adhered to a substrate and then elasticized or shrunk, for example with the application of heat; such that elastic constrictive forces are imparted to the substrate. In one particular embodiment, for example, the leg elastic members 58 include a plurality of dry-spun coalesced multifilament spandex elastomeric threads sold under the trade name LYCRA® and available from E. I. DuPont de Nemours and Company, Wilmington, Del., U.S.A.

In an alternative embodiment of the invention, all elastomeric components of the garment 20 can be located on the side panels 34 and the chassis 32 can be made of predominantly flushable or disposable materials, such that a soiled chassis can be disposed of by being flushed down a toilet or by any other suitable disposal technique and the side panels 34 can be durable such that they can be used over and over with various disposable chassis. In another alternative embodiment of the invention, both the chassis and the side panels can include elastomeric components. In yet another embodiment of the invention, the chassis can include elastomeric components while the side panels are essentially non-elastomeric. Examples of flushable and non-flushable materials are described below.

The outer cover 40 desirably includes a material that is substantially liquid impermeable, and can be elastic, stretchable or nonstretchable. The outer cover 40 can be a single layer of liquid impermeable material, but desirably includes a multi-layered laminate structure in which at least one of the layers is liquid impermeable. For instance, the outer cover 40 can include a liquid permeable outer layer and a liquid impermeable inner layer that are suitably joined together by a laminate adhesive (not shown). Suitable laminate adhesives, which can be applied continuously or intermittently as beads, a spray, parallel swirls, or the like, can be obtained from Findley Adhesives, Inc., of Wauwatosa, Wis., U.S.A., or from National Starch and Chemical Company, Bridgewater, N.J., U.S.A. The liquid permeable outer layer can be any suitable material and desirably one that provides a generally cloth-like texture and/or mating fastening component qualities. One example of such a material is a 20 gsm (grams per square meter) spunbond polypropylene nonwoven web. The outer layer may also be made of those materials of which liquid permeable bodyside liner 42 is made. While it is not a necessity for the outer layer to be liquid permeable, it is desired that it provides a relatively cloth-like texture to the wearer.

The inner layer of the outer cover 40 can be both liquid and vapor impermeable, or can be liquid impermeable and vapor permeable. The inner layer is desirably manufactured from a thin plastic film, although other flexible liquid impermeable materials may also be used. The inner layer, or the liquid impermeable outer cover 40 when a single layer, prevents waste material from wetting articles, such as bedsheets and clothing, as well as the wearer and care giver. A suitable liquid impermeable film for use as a liquid impermeable inner layer, or a single layer liquid impermeable outer cover 40, is a 0.2 millimeter polyethylene film commercially available from Huntsman Packaging of Newport News, Va., U.S.A. If the outer cover 40 is a single layer of material, it can be embossed and/or matte finished to provide a more cloth-like appearance. As earlier mentioned, the liquid impermeable material can permit vapors to escape from the interior of the disposable absorbent article, while still preventing liquids from passing through the outer cover 40. A suitable “breathable” material is composed of a microporous polymer film or a nonwoven fabric that has been coated or otherwise treated to impart a desired level of liquid impermeability. A suitable microporous film is a PMP-1 film material commercially available from Mitsui Toatsu Chemicals, Inc., Tokyo, Japan, or an XKO-8044 polyolefin film commercially available from 3M Company, Minneapolis, Minn.

The outer cover 40 can, alternatively, include breathable microporous films and laminates made out of biodegradable polymers, made using techniques known in the art. Biodegradable films may also possess properties required in flushable materials. Examples of suitable film-forming biodegradable matrix polymers include, without limitation, polylactic acid polymers (especially homopolymers); polyesters of butanediol, adipic acid, succinic acid and/or terephthalic acid; polycaprolactone polymers; and combinations thereof. An especially suitable polymer is a terpolymer of terephthalic acid, adipic acid and 1,4-butanediol, sold by BASF Corporation under the name ECOFLEX®. Filler particles used to make breathable, microporous, biodegradable films may desirably be biodegradable filler particles. Suitable biodegradable filler particles include cyclodextrin. The term “cyclodextrin” includes cyclodextrin compounds and their derivatives which retain the cyclodextrin ring-like structure in all or part of their molecular configurations.

The breathable, microporous, biodegradable film may be laminated to one or more fibrous nonwoven substrates, such as a spunbond web, meltblown web, or airlaid web, using conventional adhesive bonding or thermal bonding techniques known in the art. Biodegradable polymer compositions suitable for making films, fibers and nonwovens, for example, are taught in U.S. Pat. No. 5,939,467 issued to Wnuk et al., the contents of which are incorporated herein by reference.

The liquid permeable bodyside liner 42 is illustrated as overlying the outer cover 40 and absorbent assembly 44, and may but need not have the same dimensions as the outer cover 40. The bodyside liner 42 is desirably compliant, soft feeling, and non-irritating to the child's skin. Further, the bodyside liner 42 can be less hydrophilic than the absorbent assembly 44, to present a relatively dry surface to the wearer and permit liquid to readily penetrate through its thickness. As mentioned, the bodyside liner 42 can be made of a mating fastening component material to eliminate the need for separately attached mating fastening components.

The bodyside liner 42 can be manufactured from a wide selection of web materials, such as synthetic fibers (for example, polyester or polypropylene fibers), natural fibers (for example, wood or cotton fibers), a combination of natural and synthetic fibers, porous foams, reticulated foams, apertured plastic films, or the like. Various woven and nonwoven fabrics can be used for the bodyside liner 42. For example, the bodyside liner can be composed of a meltblown or spunbonded web of polyolefin fibers. The bodyside liner can also be a bonded-carded web composed of natural and/or synthetic fibers. The bodyside liner can be composed of a substantially hydrophobic material, and the hydrophobic material can, optionally, be treated with a surfactant or otherwise processed to impart a desired level of wettability and hydrophilicity. For example, the material can be surface treated with about 0.45 weight percent of a surfactant mixture including AHCOVEL® N-62 from Uniqema, Inc., a division of ICI of New Castle, Del., and GLUCOPON® 220UP from Cognis Corp. of Ambler, Pa., in an active ratio of 3:1. The surfactant can be applied by any conventional means, such as spraying, printing, brush coating or the like. The surfactant can be applied to the entire bodyside liner 42 or can be selectively applied to particular sections of the bodyside liner, such as the medial section along the longitudinal centerline.

A suitable liquid permeable bodyside liner 42 is a nonwoven bicomponent web having a basis weight of about 27 gsm. The nonwoven bicomponent can be a spunbond bicomponent web, or a bonded carded bicomponent web. Suitable bicomponent staple fibers include a polyethylene/polypropylene bicomponent fiber available from CHISSO Corporation, Osaka, Japan. In this particular bicomponent fiber, the polypropylene forms the core and the polyethylene forms the sheath of the fiber. Other fiber orientations are possible, such as multi-lobe, side-by-side, end-to-end, or the like. While the outer cover 40 and bodyside liner 42 can include elastomeric materials, it can be desirable in some embodiments for the composite structure to be generally inelastic, where the outer cover, the bodyside liner and the absorbent assembly include materials that are generally not elastomeric.

Alternatively, the body side liner 42 can include a urine-insoluble, water-soluble material, thereby rendering the body side liner 42 flushable. One example of such a material is a temperature-dependent, urine-insoluble, water-soluble material as described in U.S. Pat. No. 5,509,913 issued to Richard Yeo, incorporated herein by reference. The material can suitably include any of the following polymers: polyvinyl methyl ether, polyethyl oxazoline, polyvinyl pyrrolidone, hydroxypropyl cellulose, and polyvinyl alcohol having a percent hydrolysis of less than about 75%. A preferred polymer is polyvinyl alcohol, available under the trade name GOHSENOL® from Nippon Synthetic Chemical Industry Co., Ltd., of Osaka, Japan, with suitable grades including KZ-06, LL-02, and KH-17. Any of these polymers can be used in combination with a sulfate, citrate, phosphate, or chromate salt anion to make the polymer insoluble in body fluids above 25 degrees Celsius but soluble in tap water below 25 degrees Celsius. Thus, when the garment 20 is worn the garment remains intact, but when the chassis 32 is flushed down a toilet, the urine-insoluble, water-soluble material dissipates. This solubility temperature can be adjusted chemically.

The absorbent assembly 44 (FIG. 3) is positioned between the outer cover 40 and the bodyside liner 42, which components can be joined together by any suitable means, such as adhesives, as is well known in the art. The absorbent assembly 44 can be any structure which is generally compressible, conformable, non-irritating to the child's skin, and capable of absorbing and retaining liquids and certain body wastes. The absorbent assembly 44 can be manufactured in a wide variety of sizes and shapes, and from a wide variety of liquid absorbent materials commonly used in the art. For example, the absorbent assembly 44 can suitably include a matrix of hydrophilic fibers, such as a web of cellulosic fluff, mixed with particles of a high-absorbency material commonly known as superabsorbent material. High absorbency material can be provided in any form known in the art, including but not limited to particles, fibers, foams and films. Biodegradable high absorbency materials are taught, for example, in U.S. Pat. No. 4,944,734 issued to Wallach, U.S. Pat. No. 4,952,550 issued to Wallach et al., and U.S. Pat. No. 6,063,914 issued to Wolf et al., each of which is hereby incorporated by reference.

In a particular embodiment, the absorbent assembly 44 includes a matrix of cellulosic fluff, such as wood pulp fluff, and superabsorbent hydrogel-forming particles. The wood pulp fluff can be exchanged with synthetic, polymeric, meltblown fibers or with a combination of meltblown fibers and natural fibers. The superabsorbent particles can be substantially homogeneously mixed with the hydrophilic fibers or can be nonuniformly mixed. The fluff and superabsorbent particles can also be selectively placed into desired zones of the absorbent assembly 44 to better contain and absorb body exudates. The concentration of the superabsorbent particles can also vary through the thickness of the absorbent assembly 44. Alternatively, the absorbent assembly 44 can include a laminate of fibrous webs and superabsorbent material or other suitable means of maintaining a superabsorbent material in a localized area.

Suitable superabsorbent materials can be selected from natural, synthetic, and modified natural polymers and materials. The superabsorbent materials can be inorganic materials, such as silica gels, or organic compounds, such as crosslinked polymers. Suitable superabsorbent materials are available from various commercial vendors, such as Dow Chemical Company located in Midland, Mich., U.S.A., and Stockhausen GmbH & Co. KG, D-47805 Krefeld, Federal Republic of Germany. Typically, a superabsorbent material is capable of absorbing at least about 15 times its weight in water, and desirably is capable of absorbing more than about 25 times its weight in water.

In one embodiment, the absorbent assembly 44 is generally rectangular in shape, and includes a blend of wood pulp fluff and superabsorbent material. One preferred type of fluff is identified with the trade designation CR1654, available from U.S. Alliance, Childersburg, Ala., U.S.A., and is a bleached, highly absorbent sulfate wood pulp containing primarily soft wood fibers. As a general rule, the superabsorbent material is present in the absorbent assembly 44 in an amount of from about 5 to about 90 weight percent based on total weight of the absorbent assembly. The absorbent assembly 44 suitably has a density within the range of about 0.10 to about 0.50 grams per cubic centimeter. The absorbent assembly 44 may or may not be wrapped or encompassed by a suitable tissue wrap that maintains the integrity and/or shape of the absorbent assembly.

The absorbent chassis 32 can also incorporate other materials that are designed primarily to receive, temporarily store, and/or transport liquid along the mutually facing surface with the absorbent assembly 44, thereby maximizing the absorbent capacity of the absorbent assembly. One suitable material is referred to as a surge layer (not shown) and includes a material having a basis weight of about 50 to about 120 grams per square meter, and including a through-air-bonded-carded web of a homogenous blend of 60 percent 3 denier type T-256 bicomponent fiber including a polyester core/polyethylene sheath and 40 percent 6 denier type T-295 polyester fiber, both commercially available from Kosa Corporation of Salisbury, N.C., U.S.A.

Materials suitable for a flushable absorbent assembly 44 can include a water-dispersible material such as a water-dispersible polyethylene-oxide resin. The polyethylene-oxide, or other water-dispersible material, can be coated on one side, preferably a side facing the outer cover 40, with a thin, weak layer of a barrier material, such as an amorphous polyalphaolefin or a poly-caprolactone. The barrier material can be applied to the water-dispersible material either as a continuous coating or a closely-spaced discontinuous coating, thereby providing enough coverage to repel liquids while maintaining a weak enough structure to be dispersible upon contact with a considerable amount of water such as when flushed down a toilet.

In the case of a discontinuous coating of the barrier material, the spaces between the coating spots must be close enough that water is precluded by capillary forces from flowing between the spots out of the underlying substrate. Desirably, the barrier coating is a polyalphaolefin having a melt viscosity of about 400 to about 10,000 cps at 190 degrees Celsius. Suitable polymers include, but are not limited to, low molecular weight, amorphous ethylene-propylene copolymers. Particularly suitable polymers are manufactured by the U.S. Rexene Company under the tradename REXTAC®.

Water-dispersible films coated with a barrier layer are described in U.S. Pat. Nos. 4,372,311; 5,283,090; and 5,110,390, all of which are incorporated herein by reference. Other examples of water-dispersible materials include, without limitation, ethylene oxide/propylene oxide copolymers, polymethacrylic acid, polymethacrylic acid copolymers, polyvinyl alcohol, poly(2-ethyl oxazoline), polyvinyl methyl ether, polyvinyl pyrrolidone/vinyl acetate copolymers, methyl cellulose, ethyl cellulose, hydroxypropyl cellulose, hydroxypropyl methyl cellulose, ethyl hydroxyethyl cellulose, methyl ether starch, poly (n-isopropyl acrylamide), poly N-vinyl caprolactam, polyvinyl methyl oxazolidone, poly (2-isopropyl-2-oxazoline), poly (2,4-dimethyl-6-triazinyl ethylene), and combinations thereof. Examples of barrier coating materials include, without limitation, polyethylene, ethylene/vinyl acetate copolymers, polypropylene, polyesters and other water-insoluble thermoplastic resins, and combinations thereof. The barrier coating must be thin or weak enough that it ruptures or otherwise disperses when in contact with a considerable amount of water, such as when flushed down a toilet.

As noted previously, the illustrated training pant 20 has a removable side panel 34 disposed on each side of the absorbent chassis 32. These transversely opposed side panels 34 are releasably attached to the front region 22 and to the back region 24 of the chassis 32. More particularly, as shown in FIGS. 1 and 8, a pair of fastening components 82 can be permanently bonded to either an inner surface 29 or an outer surface 31 of each side panel 34 adjacent each distal edge 68 of the side panel 34, and a pair of mating fastening components 84 can be permanently bonded to either the inner surface 28 or the outer surface 30 of both the front region 22 and the back region 24 of the chassis 32, or either the inner surface 28 or the outer surface 30 of the chassis 32 can include mating fastening material 88. FIG. 1 shows an embodiment in which the fastening components 82 are bonded to the inner surface 29 of each side panel 34 and the mating fastening components 84 are bonded to the outer surface 30 of the chassis 32. FIG. 8 shows an embodiment in which the fastening components 82 are bonded to the outer surface 31 of each side panel 34 and the inner surface 28 of the chassis 32 includes mating fastening material 88. The fastening components 82 and the mating fastening components 84 may be attached to the side panels 34 and the chassis 32 using attachment means known to those skilled in the art such as adhesive, thermal or ultrasonic bonding.

In particular embodiments for improved fit and appearance, the side panels 34 desirably have an average length dimension measured parallel to the longitudinal axis 48 that is about 20 percent or greater, and particularly about 25 percent or greater, of the overall length dimension of the absorbent article, also measured parallel to the longitudinal axis 48. For example, in training pants having can overall length dimension of about 54 centimeters, the side panels 34 desirably have an average length dimension of about 10 centimeters or greater, such as about 15 centimeters. A waist end edge 72 of each side panel 34 can suitably be relatively straight across in the transverse direction while the leg end edge 70 of each side panel 34 can suitably have a curvature, as shown in FIGS. 4-7, to allow the leg opening 52 to conform about a wearer's leg.

The side panels 34 desirably include an elastic material capable of stretching in a direction generally parallel to the transverse axis 49 of the training pant 20. The entire side panel 34, or just a portion of the side panel 34, can be elastomeric. Each of the side panels 34 can include one or more individual, distinct pieces of material in addition to the fastening components 82, as shown in FIGS. 6, 7 and 10. In the embodiment shown in FIG. 6, for example, each side panel 34 can include first and second longitudinal side panel portions 90, 92 that are joined at a seam, with at least one of the portions 92 including an elastomeric material. Or alternatively, as shown in FIG. 7, non-elastomeric first and second longitudinal side panel portions 94, 96 that can include fastening material incorporated therein can be located along the distal edges 68 of the side panels 34 with a third, longitudinal, elastomeric, side panel portion 98 therebetween. Most desirably, each side panel 34 is elastomeric from the waist end edge 72 to the leg end edge 70.

Suitable elastic materials, as well as one described process of incorporating elastic side panels into a training pant, are described in the following U.S. Pat. No.: 4,940,464 issued Jul. 10, 1990 to Van Gompel et al.; U.S. Pat. No. 5,224,405 issued Jul. 6, 1993 to Pohjola; U.S. Pat. No. 5,104,116 issued Apr. 14, 1992 to Pohjola; and U.S. Pat. No. 5,046,272 issued Sep. 10, 1991 to Vogt et al.; all of which are incorporated herein by reference. In particular embodiments, the elastic material includes a stretch-thermal laminate (STL), a neck-bonded laminated (NBL), a reversibly necked laminate, or a stretch-bonded laminate (SBL) material. Methods of making such materials are well known to those skilled in the art and described in U.S. Pat. No. 4,663,220 issued May 5, 1987 to Wisneski et al.; U.S. Pat. No. 5,226,992 issued Jul. 13, 1993 to Morman; and European Patent Application No. EP 0 217 032 published on Apr. 8, 1987 in the names of Taylor et al.; all of which are incorporated herein by reference. Alternatively, the side panel material may include other woven or nonwoven materials, such as those described above as being suitable for the outer cover 40 or bodyside liner 42, or stretchable but inelastic materials.

In an alternative embodiment of the invention, the side panels 34 can include a wipe material, or material that renders the side panels 34 suitable for use as wipes. Thus, when a care giver changes a wearer's disposable absorbent garment, the side panels 34 can be used as wipes rather than requiring a separate package of wipes. In yet another alternative embodiment of the invention, the side panels 34 can each include at least one tearable, non-refastenable seam running longitudinally along the length of the side panels 34 such that the garment 20 can be removed from a wearer by tearing along the seam to loosen the fit of the garment.

As mentioned, the training pant 20 according to the present invention includes a number of fastening components 82 and/or fastening material 86 and a number of mating fastening components 84 and/or mating fastening material 88 for securing the training pant about the waist of the wearer. The fastening components 82, or fastening material 86, are adapted to refastenably connect to the mating fastening components 84, or mating fastening material 88. In one embodiment, one surface of each of the fastening components 82, or fastening material 86, includes a plurality of engaging elements that project from that surface. The engaging elements of these fastening components 82, or fastening material 86, are adapted to repeatedly engage and disengage the engaging elements of the mating fastening components 84, or mating fastening material 88.

In one particular embodiment, the fastening components 82 each include hook type fasteners and the mating fastening components 84 each include complementary loop type fasteners. In another particular embodiment, the fastening components 82 each include loop type fasteners and the mating fastening components 84 each include complementary hook type fasteners. In yet another embodiment, half of the fastening components 82 and half of the mating fastening components 84 can include hook type fasteners, while half of the fastening components and half of the mating fastening components can include loop type fasteners. In still another embodiment, the side panels 34 can be made entirely of fastener component material, i.e., either hook material or loop material. The fastening components 82 and the mating fastening components 84 are desirably rectangular, although they may alternatively be square, round, oval, curved, discontinuous or any other suitable shape.

In another embodiment, the nonwoven web in the outer cover 40 can be constructed of a material that is suitable for use as a loop-type fastening material, thereby eliminating the need for separate loop-type mating fastening components 84, and the fastening components 82 on the side panels 34 can be hook-type fastening components. In yet another embodiment, the nonwoven web in the bodyside liner 42 can be constructed of a material that is suitable for use as a loop-type fastening material, thereby eliminating the need for separate loop-type mating fastening components 84, and the fastening components 82 on the side panels 34 can be hook-type fastening components.

Loop type fasteners typically include a fabric or material having a base or backing structure and a plurality of loop members extending upwardly from at least one surface of the backing structure. The loop material can be formed of any suitable material, such as acrylic, nylon or polyester, and can be formed by methods such as warp knitting, stitch bonding or needle punching. Suitable loop materials are available from Guilford Mills, Inc., Greensboro, N.C., U.S.A. under the trade designation No. 36549. Flushable loop-type mating fastening components 84 may be made of any of the flushable materials described above in reference to the flushable outer cover 40, the flushable body side liner 42 and the flushable absorbent assembly 44.

Hook type fasteners typically include a fabric or material having a base or backing structure and a plurality of hook members extending upwardly from at least one surface of the backing structure. In contrast to the loop type fasteners which desirably include a flexible fabric, the hook material advantageously includes a resilient material to minimize unintentional disengagement of the fastener components as a result of the hook material becoming deformed and catching on clothing or other items. The term “resilient” as used herein refers to an interlocking material having a predetermined shape and the property of the interlocking material to resume the predetermined shape after being engaged and disengaged from a mating, complementary interlocking material. Suitable hook material can be molded or extruded of nylon, polypropylene or another suitable material. Suitable single-sided hook materials for the fastening components 82 or the mating fastening components 84 are available from Velcro Industries B.V., Amsterdam, Netherlands or affiliates thereof, and are identified as Velcro HTH-829 with a uni-directional hook pattern and having a thickness of about 0.089 millimeters (3.5 mils) and HTH-851 with a uni-directional hook pattern and having a thickness of about 0.051 millimeters (2 mils). Flushable hook-type mating fastening components 84 may be made of any of the flushable materials described above in reference to the flushable outer cover 40, the flushable body side liner 42 and the flushable absorbent assembly 44.

With particular reference to FIG. 9, a plurality of garment assemblies 108 with longitudinal axes aligned in a cross direction is shown. A method of making the garment 20 of the invention can be carried out with either the transverse (FIG. 9) or longitudinal (FIG. 10) product axes in the machine direction. The garment assembly 108 includes a lamination of films and nonwovens, including a bodyside liner 42 and an outer cover 40, together with waist elastics 54 and 56 (optional), leg elastics 58 (optional), and an absorbent assembly 44, as described above, placed on a vacuum device (not shown). Adjacent garment assemblies 108 are joined along the planned locations of the transversely opposed distal edges 36. The exposed surface of the garment assembly 108 shown in FIG. 9 is the inner surface 28 of the chassis 32.

In making the garment 20 of the invention in the cross direction, two mating fastening components 84 can be bonded to the back region 24 of the chassis 32 and two mating fastening components 84 can be bonded to the front region 22 of the chassis 32 of each garment assembly 108 near the planned locations of the distal edges 36, with a spacing 81 of roughly 0.25-1.0 inch between mating fastening components 84 on adjacent garment assemblies 108. Alternatively, a single, oversized mating fastening component 84 can be placed overlapping the planned locations of the distal edges 36 of two adjacent assemblies 108, to be separated into two mating fastening components 84 at the product cut-off stage. The mating fastening components 84 may include hook type fasteners or loop type fasteners, as described above, and can be bonded to the assembly 108 with ultrasonic, thermal, or adhesive bonds, or other means. As yet another alternative, the nonwoven web of the outer cover 40 or of the bodyside liner 42 can be constructed of a material that is suitable for use as a loop-type fastening material 88, thereby eliminating the need for separate mating fastening components 84 bonded to the chassis 32. As still another alternative, the side panels 34 can be constructed of a material that is suitable for use as a hook-type fastening material 86, thereby eliminating the need for separate fastening components 82 bonded to the side panels 34.

After the mating fastening components 84 have been bonded to the garment assemblies 108 or if the material of the chassis 32 is suitable for use as a fastening material, then two side panels 34, each having two fastening components 82 bonded thereon, or each made of a fastening material, are folded at least once, i.e. C-folded, and placed on the mating fastening components 84 on the inner surface 28 of the chassis 32 of each garment assembly 108 near the planned locations of the distal edges 36, also with a spacing 81 of roughly 0.25-1.0 inch between mating fastening components 84 on adjacent garment assemblies 108.

All of the joined garment assemblies 108 are then folded longitudinally, thereby aligning the mating fastening components 84 with the folded fastening components 82. FIG. 11 shows a top view of a waist edge portion of two adjacent garment assemblies 108 at this point of the manufacturing process. Adjacent garment assemblies 108 are then separated along cut line 110, shown in FIG. 11, thereby forming discrete products with removable side panels 34. The side panels 34 and the chassis 32 can be unfastened and refastened on either the front region 22 of the garment 20 or the back region 24 of the garment 20, on either the left side or the right side of the garment.

As mentioned, the garment of the invention can also be made in the machine direction, as shown in FIG. 10. In making the garment of the invention in the machine direction, two mating fastening components 84 can be bonded to the back region 24 of the chassis 32 and two mating fastening components 84 can be bonded to the front region 22 of the chassis 32 of each garment assembly 108. Alternatively, two oversized strips of mating fastening components can be applied to both the front region 22 and the back region 24, overlapping the planned locations of the front waist edge 38 and the back waist edge 39 of adjacent assemblies 108, to be separated at the product cut-off stage. As yet another alternative, the nonwoven web of the outer cover 40 or of the bodyside liner 42 can be constructed of a material that is suitable for use as a loop-type fastening material, thereby eliminating the need for separate mating fastening components 84 bonded to the chassis 32.

After the mating fastening components 84 have been bonded to the garment assemblies 108 in the machine direction process (FIG. 10) or if the material of the chassis 32 is suitable for use as a fastening material, then adjacent assemblies 108 can be separated at the planned locations of the front waist edge 38 and the back waist edge 39 of adjacent assemblies 108 along cut lines 110. Two side panels 34, each with a pair of fastening components 82 bonded thereon or each made of a fastening material, can be folded at least once, i.e. C-folded, and placed on the front region 22, or back region 24, near the distal edges 36 of the chassis 32. Alternatively, prior to separating adjacent garment assemblies 108, two oversized side panels 34, each with a pair of fastening components 82 bonded thereto, and roughly twice as long as the typical side panels 34 and fastening components 82, can be folded and placed on the mating fastening components 84 of two adjacent garment assemblies 108, overlapping the planned locations of the front waist edge 38 and the back waist edge 39 of the adjacent garment assemblies 108, to be separated into two side panels 34 and two fastening components 82 at the product cut-off stage. In this alternative method, the oversized, folded side panels 34 are applied to every other planned location of the front waist edge 38 and the back waist edge 39 of adjacent garment assemblies 108.

Once the adjacent garment assemblies 108 have been separated from one another, each individual garment assembly 108 is folded longitudinally, thereby aligning the fastening components 82 with the mating fastening components 84 on the folded side panels 34, as shown in FIGS. 8 and 12.

Various embodiments of the invention include an embodiment in which the mating fastening components 84 are bonded to the outer surface 30 of the front and back regions 22, 24 of the chassis 32 and the fastening components 82 are bonded to the inner surface 29 of the side panels 34, as shown in FIGS. 1 and 13. In another embodiment of the invention, shown in FIG. 14, two of the mating fastening components 84 are bonded to the inner surface 28 of the front region 22 of the chassis 32, two of the mating fastening components 84 are bonded to the outer surface 30 of the back region 24 of the chassis 32, two of the fastening components 82 are bonded to the inner surface 29 of the side panels 34 and two of the fastening components 82 are bonded to the outer surface 31 of the side panels 34. In yet another embodiment of the invention, shown in FIG. 15, two of the mating fastening components 84 are bonded to the outer surface 30 of the front region 22 of the chassis 32, two of the mating fastening components 84 are bonded to the inner surface 28 of the back region 24 of the chassis 32, two of the fastening components 82 are bonded to the inner surface 29 of the side panels 34 and two of the fastening components 82 are bonded to the outer surface 31 of the side panels 34. In still another embodiment of the invention, the side panel material serves as a fastening material 86 and/or the chassis material serves as a mating fastening material 88 that can engage with the fastening material of the side panels, thus eliminating the need for separately attached fastening components 82 and mating fastening components 84.

The absorbent chassis 32 and the removable side panels 34 together define a refastenable product having a waist opening 50 and a pair of leg openings 52. When the fastening components 82 and the mating fastening components 84 are engaged, the refastenable product can include a pair of elastomeric side panels 34 extending from the waist opening 50 to each leg opening 52. Suitably, more than one leg elastic member 58 can partially or fully encircle each leg opening 52. Each leg elastic member 58 can extend from adjacent a distal edge 36 of the front region 22 to adjacent a distal edge 36 of the back region 24. Alternatively, all elastomeric components of the garment 20 can be included in the removable side panels 34 and all materials used in the chassis 32 can be flushable, such that the side panels 34 can be reused over and over while each soiled chassis 32 can be flushed down a toilet or disposed of in another manner such as composting.

As described herein, the various components of the absorbent garment 20 can be integrally assembled together employing various types of suitable attachment means, such as adhesive, sonic and thermal bonds or combinations thereof. The resulting product is an absorbent garment 20 having removable side panels 34 that can be unfastened or removed all together, for donning or removal of the garment at either or both sides of the garment.

It will be appreciated that details of the foregoing embodiments, given for purposes of illustration, are not to be construed as limiting the scope of this invention. Although only a few exemplary embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention, which is defined in the following claims and all equivalents thereto. Further, it is recognized that many embodiments may be conceived that do not achieve all of the advantages of some embodiments, particularly of the preferred embodiments, yet the absence of a particular advantage shall not be construed to necessarily mean that such an embodiment is outside the scope of the present invention.

We claim:

1. An absorbent garment comprising:
a chassis including a front panel and a back panel;
first and second side panels, each being releasably connected to the front and back panels, and defining with the chassis a waist opening and first and second leg openings;
wherein the first side panel includes first and second fastening components, the second side panel includes third and fourth fastening components, the front panel includes first and third mating fastening components engageable with the first and third fastening components, and the back panel includes second and fourth mating fastening components engageable with the second and fourth fastening components.

2. The absorbent garment of claim 1, wherein each of the fastening components comprises a hook material and each of the mating fastening components comprises a loop material.

3. The absorbent garment of claim 1, wherein each of the fastening components comprises a loop material and each of the mating fastening components comprises a hook material.

4. The absorbent garment of claim 1, wherein two of the fastening components and two of the mating fastening components each comprise a hook material, and two of the fastening components and two of the mating fastening components each comprise a loop material.

5. The absorbent garment of claim 1, wherein the first and second side panels comprise a wipe material.

6. The absorbent garment of claim 1, wherein the chassis comprises an elastic material.

7. The absorbent garment of claim 6, wherein the first and second side panels comprise an elastic material.

8. The absorbent garment of claim 6, wherein the first and second side panels comprise an inelastic material.

9. The absorbent garment of claim 1, wherein the chassis comprises an inelastic material and the first and second side panels comprise an elastic material.

10. The absorbent garment of claim 1, wherein the chassis comprises a disposable material.

11. The absorbent garment of claim 10, wherein the first and second side panels comprise a disposable material.

12. The absorbent garment of claim 10, wherein the first and second side panels comprise a durable material.

13. The absorbent garment of claim 10, wherein the chassis comprises a flushable outer cover, a flushable body side liner, and a flushable absorbent assembly.

14. The absorbent garment of claim 1, wherein the first and second side panels each comprise at least two pieces of material bonded to one another.

15. The absorbent garment of claim 14, wherein at least one of the pieces of material is elastomeric and at least one of the pieces of material is non-elastomeric.

16. The absorbent garment of claim 14, wherein a first non-elastomeric piece of material is bonded to a second elastomeric piece of material and a third non-elastomeric piece of material is bonded to the second elastomeric piece of material.

17. The absorbent garment of claim 1, comprising a diaper.

18. The absorbent garment of claim 1, comprising child training pants.

19. The absorbent garment of claim 1, comprising an adult incontinence garment.

20. The absorbent garment of claim 1, comprising swim wear.

21. An absorbent garment comprising:
an absorbent chassis including a front panel and a back panel;
first and second elastomeric side panels releasably connected to the front and back panels and defining with the chassis a waist opening and first and second leg openings;
a first refastenable seam extending from the waist opening to the first leg opening between the first side panel and the front panel;
a second refastenable seam extending from the waist opening to the first leg opening between the first side panel and the back panel;
a third refastenable seam extending from the waist opening to the second leg opening between the second side panel and the front panel; and
a fourth refastenable seam extending from the waist opening to the second leg opening between the second side panel and the back panel.

22. The absorbent garment of claim 21, wherein the first seam comprises a fastening material on an inner surface adjacent a distal edge of the front panel and a mating fastening material on a corresponding outer surface adjacent a distal edge of the first side panel.

23. The absorbent garment of claim 21, wherein the first seam comprises a fastening material on an outer surface adjacent a distal edge of the front panel and a mating fastening material on a corresponding inner surface adjacent a distal edge of the first side panel.

24. The absorbent garment of claim 21, wherein the second seam comprises a fastening material on an inner surface adjacent a distal edge of the back panel and a mating fastening material on a corresponding outer surface adjacent a distal edge of the first side panel.

25. The absorbent garment of claim 21, wherein the second seam comprises a fastening material on an outer surface adjacent a distal edge of the back panel and a mating fastening material on a corresponding inner surface adjacent a distal edge of the first side panel.

26. The absorbent garment of claim 21, wherein the third seam comprises a fastening material on an inner surface adjacent a distal edge of the front panel and a mating fastening material on a corresponding outer surface adjacent a distal edge of the second side panel.

27. The absorbent garment of claim 21, wherein the third seam comprises a fastening material on an outer surface adjacent a distal edge of the front panel and a mating fastening material on a corresponding inner surface adjacent a distal edge of the second side panel.

28. The absorbent garment of claim 21, wherein the fourth seam comprises a fastening material on an inner surface adjacent a distal edge of the back panel and a mating fastening material on a corresponding outer surface adjacent a distal edge of the second side panel.

29. The absorbent garment of claim 21, wherein the fourth seam comprises a fastening material on an outer surface adjacent a distal edge of the back panel and a mating fastening material on a corresponding inner surface adjacent a distal edge of the second side panel.

30. The absorbent garment of claim 21, wherein the absorbent chassis further comprises an outer cover that functions as a loop material.

31. The absorbent garment of claim 21, wherein the absorbent chassis further comprises a flushable outer cover.

32. The absorbent garment of claim 21, wherein the absorbent chassis further comprises a flushable body side liner.

33. The absorbent garment of claim 21, wherein the absorbent chassis further comprises a flushable absorbent assembly.

34. An absorbent garment comprising:
a chassis including a front panel and a back panel, the front and back panels each having at least two fastening components bonded thereon, and partially defining a waist opening and first and second leg openings;
a first side panel including a substrate and at least two mating fastening components extending from the waist opening to the first leg opening, at least one of the mating fastening components releasably connected to at least one of the fastening components on one of the front and back panels; and
a second side panel including a substrate and at least two mating fastening components extending from the waist opening to the second leg opening, at least one of the mating fastening components releasably connected to at least one of the fastening components on one of the front and back panels.

35. The absorbent garment of claim 34, wherein the fastening components comprise a hook material and the mating fastening components comprise a loop material.

36. The absorbent garment of claim 34, wherein the fastening components comprise a loop material and the mating fastening components comprise a hook material.

37. The absorbent garment of claim 34, wherein the first and second side panels comprise an elastic material.

38. The absorbent garment of claim 34, wherein the first and second side panels comprise a wipe material.

39. The absorbent garment of claim 34, wherein the first and second side panels each comprise at least one tearable, non-refastenable seam.

40. The absorbent garment of claim 34, wherein the chassis comprises a flushable outer cover, a flushable body side liner, and a flushable absorbent assembly.
